(19) United States
(12) Patent Application Publication
(10) Pub. No.: US 2020/0275862 A1
(43) Pub. Date: Sep. 3, 2020

(54) MULTIPLE PHYSICAL CONDITIONS EMBODIED IN BODY PART IMAGES TO GENERATE AN ORTHOTIC

(71) Applicant: Wiivv Wearables Inc., Vancouver (CA)

(72) Inventors: Carly M. Fennell, Vancouver (CA); Amy J. Vanden Eynde, Vancouver (CA); Michael C. Salmon, West Vancouver (CA); Colin M. Lawson, Calgary (CA); Christopher W. Bellamy, Vancouver (CA); Brett D. Ritter, Santa Barbara, CA (US); Shamil Hargovan, San Jose, CA (US); Myat Thu, Burnaby (CA); Lino Evgueni Coria Mendoza, Port Moody (CA); Hsiao Ting Lin, Coquitlam (CA)

(21) Appl. No.: 16/290,784

(22) Filed: Mar. 1, 2019

(51) Int. Cl. A61B 5/107; A43D 1/02; A61B 5/00; A61F 5/01

(52) U.S. Cl. CPC A61B 5/1074; A43D 1/02; G01B 11/24; A61F 5/01; A61B 5/1072; A61B 5/0064

(57) ABSTRACT

Disclosed is a technique for use in generating and delivering 3-D printed wearables via the use of body image data where the body is in under multiple physical conditions. Some embodiments include further body part data including videos of sequences of motion, static images, both 2-D and 3-D modeling, and user preference. Users take videos and/or photos of parts of their body as input into a custom wearable generation application. The body photos are subjected to a precise computer vision (CV) process to determine specific measurements of the user's body and the stresses.

### TECHNICAL FIELD

This disclosure relates to 3-D digital modeling and, more particularly, to computer interpretation of image data to generate 3-D digitals of orthotics.

### BACKGROUND

People tend to like products that are customized for them more than generic products. Despite interest in customized products, consumers are less inclined toward customization if obtaining personal specifications is bothersome. Physically measuring oneself is bothersome. Using complex equipment to measure oneself either by oneself, or at the office of a related professional is also bothersome. Most people carry smartphones that include digital cameras and a connection to the Internet. 3-D printers and other programmable manufacturing apparatus enable the generation of custom physical wearables from digital models of users.

### BRIEF DESCRIPTION OF THE DRAWINGS

FIG. 1 is a block diagram illustrating a system for the generation of customized 3-D printed wearables.

FIG. 2 is a flowchart illustrating a process for performing computer vision on collected images of a user in multiple physical conditions.

FIG. 3 is an illustration of a coordinate graph including a collection of X,Y locations along a body curve.

FIG. 4 is an illustration of three physical conditions for a foot arch.

FIG. 5 is an illustration of a biomechanical analysis on image data of a foot under multiple physical conditions.

FIG. 6 is a flowchart illustrating a process for performing computer vision on video input including a sequence of motion having multiple physical conditions.

FIG. 7A is an illustration depicting body kinematics during an active sequence of motion.

FIG. 7B is an illustration of two physical states of a hand.

FIG. 7C is an illustration of multiple physical states of a knee.

FIG. 7D is an illustration of a sequence of motion of an arm and torso.

FIG. 8 is an illustration of a key point analysis upon a breast during a sequence of motion.

FIG. 9 is a flowchart illustrating wearable generation including simultaneous computer vision and machine learning processes.

### DETAILED DESCRIPTION

By using computer vision techniques, two-dimensional (2-D) and/or three-dimensional (3-D) digital models can be constructed for objects found in image data. The digital models subsequently can be used for numerous activities, including generation of 3-D printed objects sized and shaped to match the objects found in the image data. For example, in some embodiments, images of a human body are used to model at least a portion of the human body, and then customized wearables can be printed for the modeled portion of the human body (e.g., footwear, headwear, undergarments, sportswear, etc.). Depending on the subject object (e.g., body part), the image data includes views of that object and key points on that object in various physical states (e.g., physical positions, weight loads, gravity states, temporal periods) in one or more data types (e.g., video frames, 2D/3D static images, inertial measurements, user preference).

In some embodiments, users are directed to use a mobile device, such as a smartphone including a camera, to take photos of some subject object (e.g., their feet). In some embodiments, different and/or multiple modes of the smartphone camera are used. For example, a given implementation may make use of static 2-D images, static 3-D images, video frames where the user's body is in motion, inertial measurements, and specified user preferences. In this case, a “static” image is one that is not associated with a series of frames in a video. In some embodiments, additional apparatus beyond a given mobile device (or smartphone) is used to collect input data of the user's body. Videos of the user's body in motion may include different poses (e.g., clenched/unclenched, different states of bearing weight, etc.) and/or may include cycles of motion (e.g., walking/jogging/running one or more strides, jumping, flexing, rotating a joint, etc.).

Key points on a target body part and/or associated body parts are attached to visual input (image data static/video). Tracking the movement of those body parts between the various images or video frames provides important data to accurately understand the user's body and the type of wearable item that person would want or need. Using machine learned models, AI, and/or heuristics, the shift/motion of the key points in various body states directs a system to generate a model of a wearable for the user. That wearable is biometrically suited for the user.

As an illustrative example, identifying how a foot arch displaces as weight is applied to it can be used to determine the amount and style of arch support a person needs. Knowing the direction and amount of force that motion of a woman's breast puts on the torso during aerobic activity can similarly be used to determine the amount and style of breast support the woman's needs.

The 3-D models generated based on the user body data are sent to a manufacturing apparatus to generate. In some embodiments, the manufacturing apparatus is a 3-D printer. 3-D printing refers to a process of additive manufacturing. In some embodiments, components are machine generated in custom sizes based off the 3-D model (e.g., laser cut cloth or machine sewed) and then assembled by hand or machine.

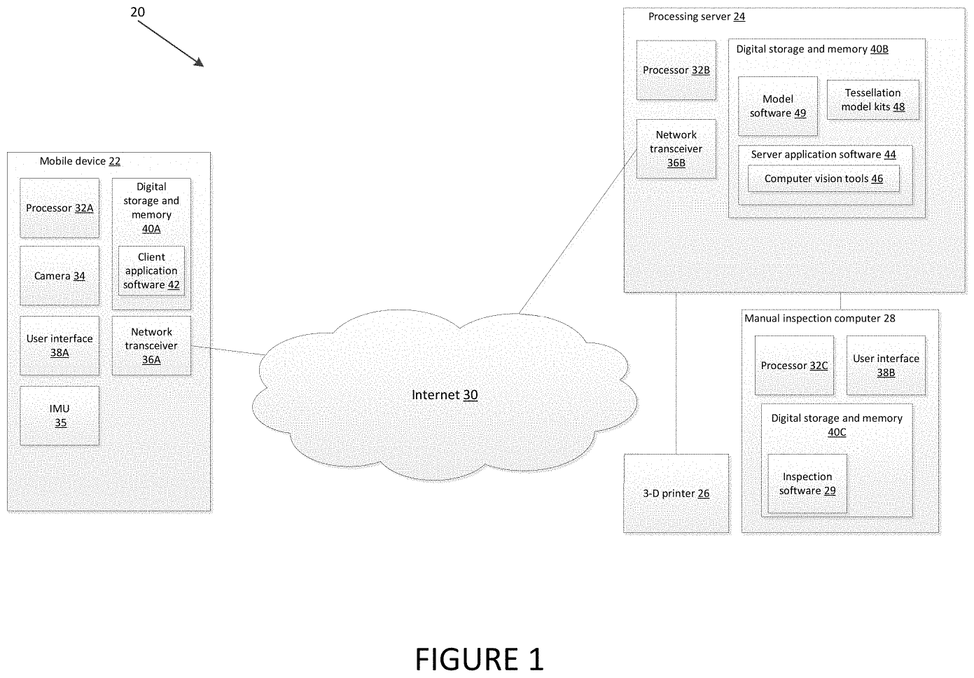

FIG. 1 is a block diagram illustrating a system 20 for the generation of customized 3-D printed wearables. Included in the system 20 is the capability for providing body part input data. Provided as a first example of such a capability in FIG. 1 is a mobile processing device (hereafter, “mobile device”) 22 that includes a digital camera 34 and is equipped to communicate over wireless network, such as a smartphone, tablet computer, a networked digital camera or other suitable known mobile devices in the art; a processing server 24; and a 3-D printer or other manufacturing apparatus 26. The system further can include a manual inspection computer 28.

The mobile device 22 is a device that is capable of capturing and transmitting images over a network, such as the Internet 30. In practice, a number of mobile devices 22 can be used. In some embodiments, the mobile device 22 is a handheld device. Examples of mobile devices 22 include a smart phone (e.g., Apple iPhone, Samsung Galaxy), a confocal microscopy body scanner, an infrared camera, an ultrasound camera, a digital camera, and a tablet computer (e.g., Apple iPad or Dell Venture 10 7000). The mobile device 22 is a processor enabled device including a camera 34, an inertial measurement unit 35, a network transceiver 36A, a user interface 38A, and digital storage and memory 40A containing client application software 42.

The camera 34 on the mobile device may be a simple digital camera or a more complex 3-D camera, scanning device, InfraRed device, or video capture device. Examples of 3-D cameras include Intel RealSense cameras or Lytro light field cameras. Further examples of complex cameras may include scanners developed by TOM-CAT Solutions, LLC (the TOM-CAT, or iTOM-CAT), adapted versions of infrared cameras, ultrasound cameras, or adapted versions of intra-oral scanners by 3Shape.

The inertial measurement unit 35 is enabled to track movement of the mobile device 22. Movement may include translation and rotation within 6 degrees-of-freedom as well as acceleration. In some embodiments, the motion tracked may be used to generate a path through space. The path through space may be reduced to a single vector having a starting point and an end point. For example, if held in the hand while running, the mobile device 22 will jostle up and down as the runner sways their arms. A significant portion of this motion is negated over the course of several strides

Simple digital cameras (including no sensors beyond 2-D optical) use reference objects of known size to calculate distances within images. Use of a 3-D camera may reduce or eliminate the need for a reference object because 3-D cameras are capable of calculating distances within a given image without any predetermined sizes/distances in the images.

The mobile device also provides a user interface 38A that is used in connection with the client application software 42. The client application software 42 provides the user with the ability to select various 3-D printed wearable products. The selection of products corresponds with camera instructions for images that the user is to capture. Captured images are delivered over the Internet 30 to the processing server 24.

The processer 32B controls the overall operation of the processing server 24. The processing server 24 receives image data from the mobile device 22. Using the image data, server application software 44 performs image processing, machine learning and computer vision operations that populate characteristics of the user. The server application software 44 includes computer vision tools 46 to aid in the performance of computer vision operations. Examples of computer vision tools 46 include OpenCV or SimpleCV, though other suitable examples are known in the art and may be programmed to identify pixel variations in digital images. Pixel variation data is implemented as taught herein to produce desired results.

In some embodiments, a user or administrative user may perform manual checks and/or edits to the results of the computer vision operations. The manual checks are performed on the manual inspection computer 28 or at a terminal that accesses processing server's 24 resources. The processing server 24 includes a number of premade tessellation model kits 48 corresponding to products that the user selects from the client application software 42. Edits may affect both functional and cosmetic details of the wearable—such edits can include looseness/tightness, and high rise/low rise fit. Edits are further stored by the processing server 24 as observations to improve machine learning algorithms. In some embodiments, modeling software 49 is used to generate models of wearables from input body data.

In some embodiments, the tessellation model kits 48 are used as a starting point from which the processing server 24 applies customizations. Tessellation model kits 48 are a collection of data files that can be used to digitally render an object for 3-D printing and to print the object using the 3-D printer 26. Common file types of tessellation model kits 48 include .3mf, .3dm, .3ds, .blend, .bvh, .c4d, .dae, .dds, .dxf, .fbx, .lwo, .lws, .max, .mtl, .obj, .skp, .stl, .tga, or other suitable file types known in the art. The customizations generate a file for use with a 3-D printer. The processing server 24 is in communication with the manufacturing apparatus 26 in order to print out the user's desired 3-D wearable. In some embodiments, tessellation files 48 are generated on the fly from the input provided to the system. The tessellation file 48 is instead generated without premade input through an image processing, computer vision, and machine learning process.

Any of numerous models of manufacturing apparatus 26 may be used by the system 20. Manufacturing apparatus 26 vary in size and type of generated wearable article. In the case where the 3-D wearable is a bra, for example, one may implement a laser cut cloth. Where the 3-D wearable is an insole, or arch support, one may implement a 3-D printer.

Users of the system may take a number of roles. Some users may be administrators, some may be intended wearers of a 3-D printed product, some users may facilitate obtaining input data for the system, and some may be agents working on behalf of any user type previously mentioned.

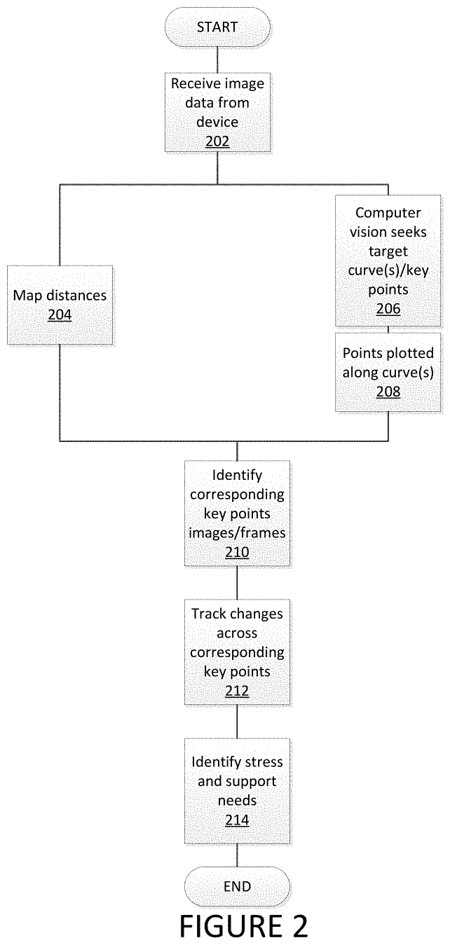

FIG. 2 is a flowchart illustrating a process for performing computer vision on collected user images in order to generate size and curvature specifications. FIG. 2 is directed to the example of a foot, though other body parts work similarly. The curves of each body part vary; the foot in this example is a complex, curved body structure. The steps of FIG. 2 in at least some embodiments are all performed by the server application software. In step 202, the processing server receives image data from the mobile device. Once received, in step 204 and 206, the processing server performs computer vision operations on the acquired image data to determine size and curvature specifications for the user's applicable body part in different states.

In step 204, the server application software analyzes the image data to determine distances between known points or objects on the subject's body part. Example distances include heel to big toe, heel to little toe, joint of big toe horizontally across, and distance from either side of the first to fifth metatarsal bones. This process entails using predetermined or calculable distances based on a reference object or calculated distances with knowledge of camera movement to provide a known distance and angle using stereoscopic images or other 3-D imaging technique. In some embodiments, the reference object can be a piece of standard size paper (such as 8.5″×11″), as mentioned above. The application software then uses known distances to calculate unknown distances associated with the user's body part based on the image.

In step 206, the processing server analyzes the image data for body part curvature and/or key points. The key points may exist both on and off the target body part. Key points that exist off the target body part are used as a control or reference point. The computer vision process seeks an expected curve or stress area associated with the body part and with the type of wearable selected. Key points and curves have corresponding data across each of the images. In each image the key point has potentially shifted based on body part movement.

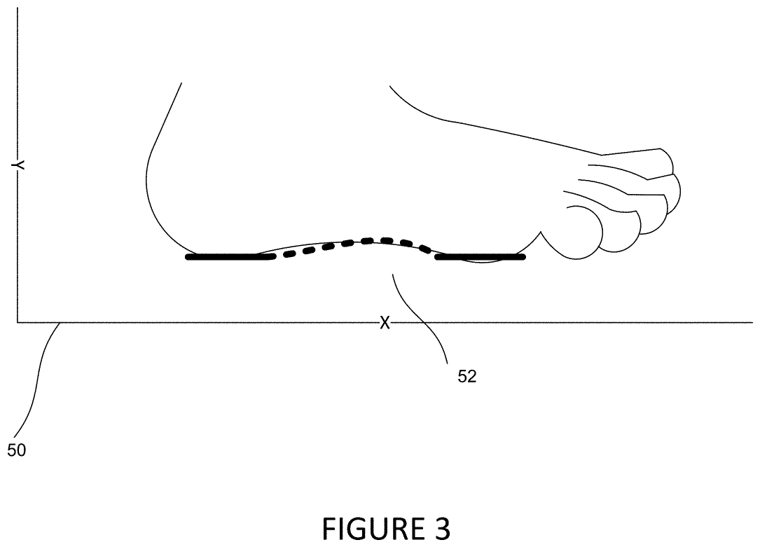

Once the curve or stress area is found, in step 208, points are plotted on the image data (either from the static frames or video frames) in a coordinate graph (see FIG. 3). Shown in FIG. 3, the coordinate graph 50 includes an X,Y location along the curve in a collection of points 52. Taken together, the collection of points 52 model the curvature of the body part (here, the arch of a foot). In some embodiments, the coordinate graph further includes a third, Z dimension. In various embodiments, the third, Z dimension is a natural part of a 3-D image, or as an added dimension in a 2-D image.

Notably, the analysis of FIG. 2 may be performed using a large trained model (with many thousands or millions of data points). In some embodiments, the analysis makes use of a heuristic to identify the curves or key points. In some embodiments, the FIG. 2 analysis is performed on an application/backend server where the computational complexity or memory footprint of the trained model is of little concern to the overall user experience.

Returning to FIG. 2, in step 210, the processing server identifies which key points correspond to one another between images/frames. Using the distance data from step 204, points from one image may be associated with corresponding points from adjoining images. For example, even if a point of skin translates vertically as weight is applied to a foot and the foot arch displaces, that point of skin is still at a similar distance from the heal and toe (by absolute values or percentage of foot length). As that area of skin shifts through a sequence of motion or physical state change, it may continue to be tracked.

In step 212, the system identifies changes to each of the corresponding points across the image data. The data regarding the shift of a given key point illustrates where stress/force is being applied to the body. In step 214, the system identifies the stress and support needs of the body part. In some embodiments, the magnitude of the force is calculated based on overall mass and vectors of movement (including distance and speed traveled). Vectors of movement indicate the regions of the body that shift and move and the direction of that movement. The system identifies a support plan based on where those regions are, and whether that portion of the body is intended to move during the specified sequence of motion or change in physical state.

A support plan includes a position of a support feature based on where stresses are being experienced, and a structure for the support feature based on the magnitude of the stress. For example, depending on whether a user's walking/jogging/running gait is one where the user plants with their heel, or their toes, support is positioned differently in an orthotic. In some embodiments, the support plan includes a rigidity factor that may vary across regions of the sole of the foot (“plantar zones”). The rigidity for each plantar zone refers to how rigid an orthotic is across various points of interface with the foot. Further, the speed of their stride will affect the magnitude of the force experienced, and thus a user with faster, heavier strides will include more padding. Additionally, in some embodiments, the manner the wearer plans to use the orthotic influences the support plan. Wearers who are runners may include a different support plan than wearers who stand in place all day. In a bra example, the magnitude of the force may influence varied bra strap configurations, bra strap thicknesses, padding thicknesses and positioning, and underwire configurations and thicknesses.

In some embodiments, the shift of the points is used generate a gait analysis. The manner in which a given person walks tends to determine the manner in which force is applied to various parts of their body. Each aspect that may be extrapolated from the shift of key points is actionable data for identifying support/stress needs of the body. A trained biometric model is applied to the extrapolated body data to determine a style of wearable to generate that addresses these stress/support needs.

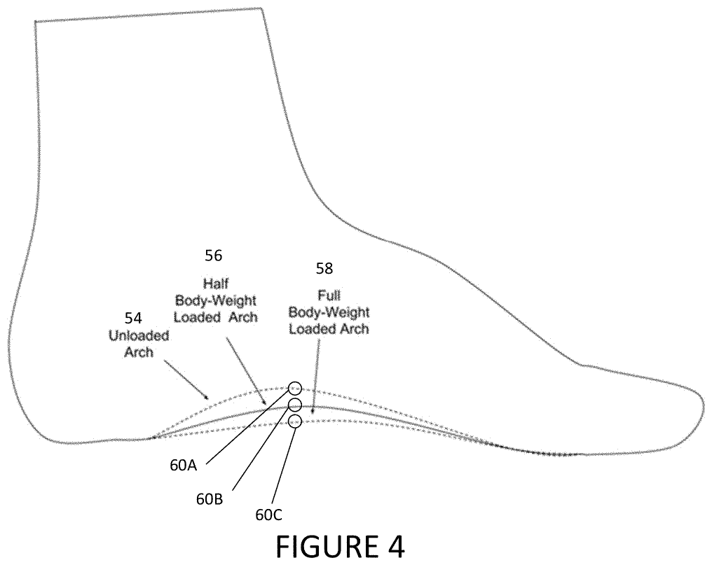

FIG. 4 is an illustration of three physical conditions for a foot arch. Physical conditions may include a number of different weight loaded states. A foot without body weight 54 has a higher arch than a foot with half of a person's weight 56, and an even higher arch than a foot having all of a person's weight 58. The body image data may exist in a number of forms including static 2-D images, static 3-D images, and video data. The video data may capture a sequence of motion such as the transition from supporting no body weight to all of a user's body weight. The camera of a mobile device captures at least two physical conditions of a given body part (such as a foot), and body modeling systems identify changes in key body points between the different physical conditions.

In each image of a given physical condition there are key points 60. The key points 60 correspond to one another across each physical condition. As pictured, key points 60A, 60B, and 60C each correspond to one another. Each of the corresponding key points 60A-C are located at the same location on the foot and have shifted based on changes in the physical condition of the foot.

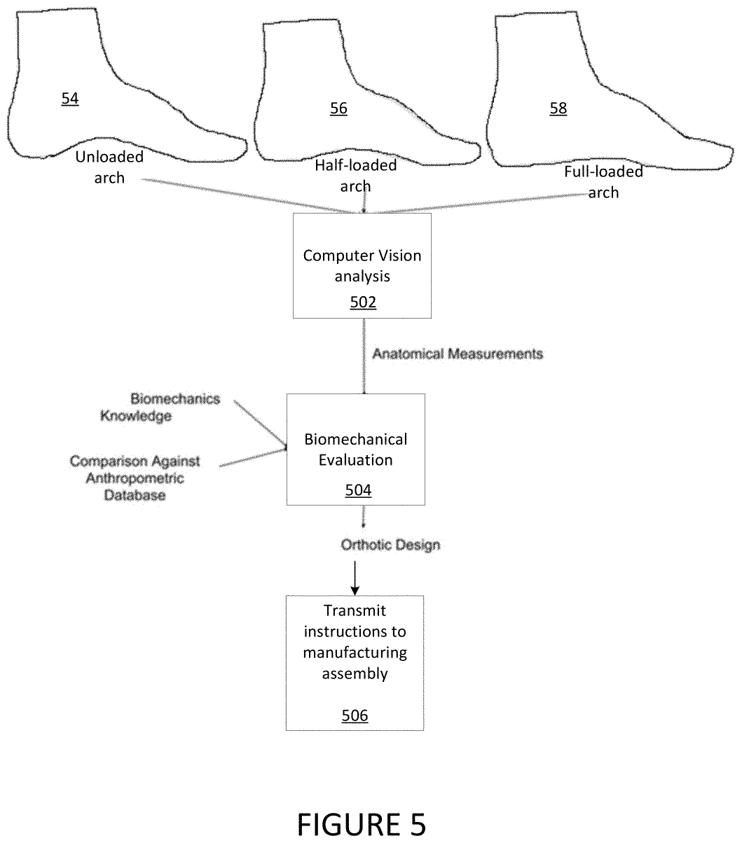

FIG. 5 is an illustration of a biomechanical analysis flowchart on image data of a foot under multiple physical conditions. The difference between the shape of a person's arches between different physical conditions is indicative of a style and degree of orthotic support. First, the system receives image data of the body part 54-58 (e.g., in this illustrative example, a foot) in multiple physical conditions.

Prior to biomechanical analysis, the system identifies what body part is subject of the analysis. Based on the target body part, a different analysis, trained machine model, and/or knowledge base is applied to the input data. For example, various types of physical conditions across the data may be used for various orthotics. Examples of physical conditions include supporting different weight loads (as shown in FIG. 4), flexed and unflexed (e.g., muscles), clenched and unclenched (e.g., a first and an open hand), at different states of gravity (e.g., various effects of gravity throughout the course of a jump), or at different temporal periods (e.g., spinal compression when the user wakes up as compared to when they return from work).

In step 502, the system performs a computer vision analysis on the image data 54-58. The computer vision analysis identifies anatomical measurements of the body part, as well as identify location of corresponding key points.

In step 504, the system performs a biomechanical evaluation of the various images. The biomechanical analysis may vary based on body part. The biomechanical analysis includes tracking the shift of the key points across the different physical conditions. As part of the biomechanical evaluation, the system may generate a model of the body part. Various embodiments of the body part model may exist as a 3-D point cloud, a set of 2-D coordinates, a set of vectors illustrating movement/shift of key points, a set of force measurements, and/or data describing the body (e.g., an estimated mass).

Based on the body part analyzed, a different biomechanical knowledge base is applied. Based on body type, a different anthropometric database is applied. For example, a user who has fallen arches in their feet uses an anthropometric database/trained model for users with fallen arches. Based on the shift of key points and the applicable anthropometric database/trained model, the system identifies a particular orthotic design (e.g., a starting tessellation kit to work from) that the user needs. The system adjusts the orthotic design for the user's specific measurements.

In step 506, the model of the wearable orthotic is transmitted towards a manufacturing apparatus such as a 3-D printer or a garment generator (e.g., procedural sewing device or automatic clothing laser cutter).

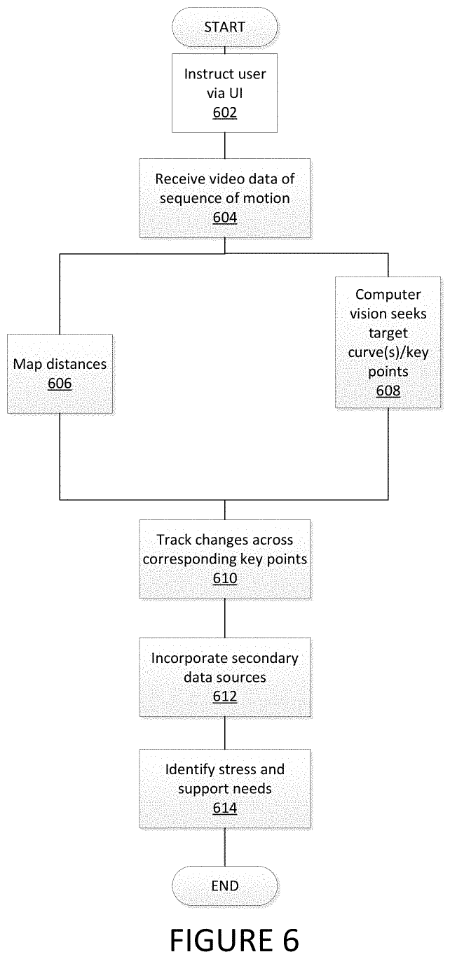

FIG. 6 is a flowchart illustrating a process for performing computer vision on video input including a sequence of motion having multiple physical conditions. The manner in which the system operates on video data input is similar to how the system operates on static frames as described with respect to FIG. 2. There are notable differences with respect to the user interface and the user experience.

In step 602, the user interface of the mobile device instructs the user how to collect the video data. In some circumstances a partner (“secondary user”) may be necessary to obtain the relevant target body part. Depending on the body part, or sequence of motion for capture, the instructions vary.

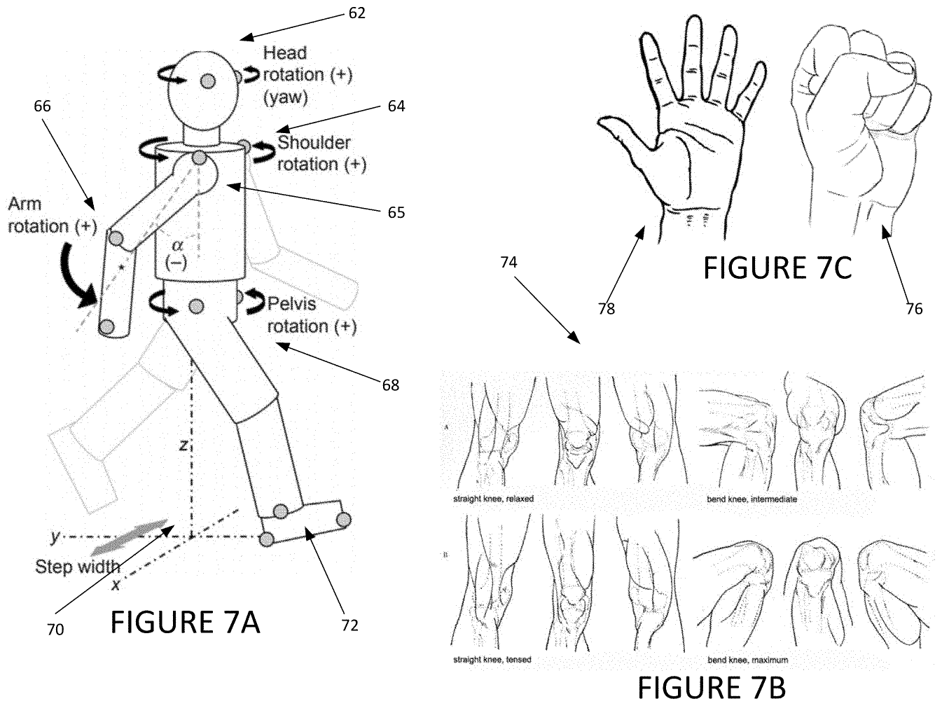

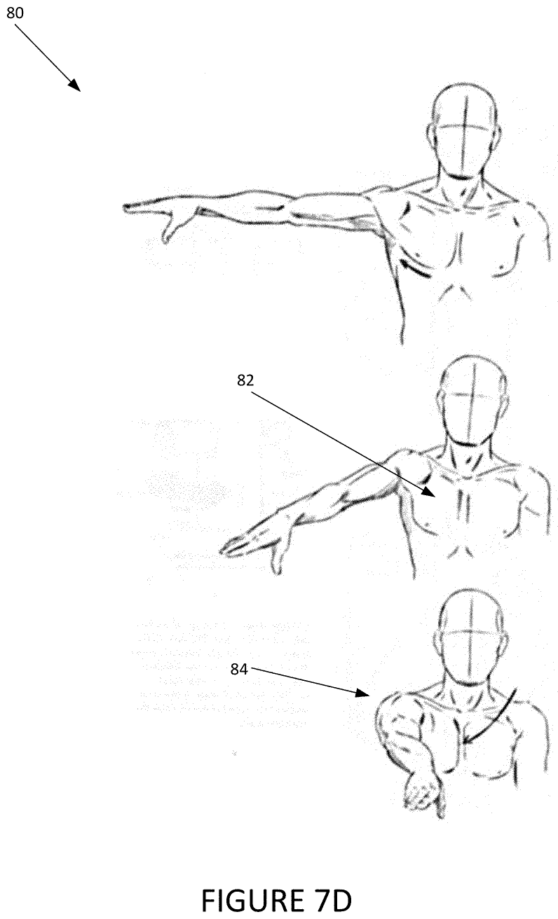

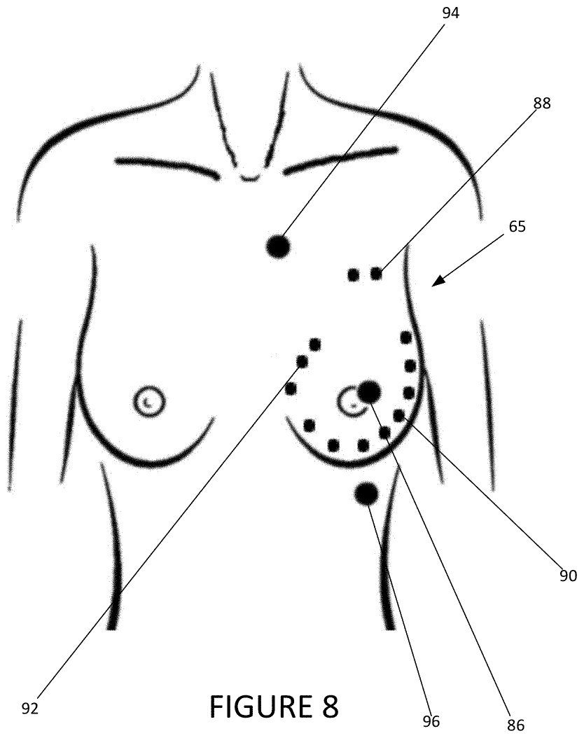

A number of sequences of motion are depicted in FIGS. 7A-D. A sequence of motion is movement of a body part (or body parts) through a number of physical conditions. FIG. 7A is an illustration depicting body kinematics during an active translational sequence of motion such as walking, running, or jogging. During translational movement, a number of body parts may be examined as target body parts. Examples depictured include head and neck rotation 62, shoulder rotation 64, breast movement 65, arm swaying 66, pelvic rotation 68, gait 70, and ankle and foot movement 72. FIG. 7B is an illustration of multiple physical states for a knee. Sequences of motion including the knee include multiple extension states and loading states 74. FIG. 7C is an illustration of two physical states for a hand, clenched 76 and extended 78. FIG. 7D is an illustration of a sequence of motion for an arm and torso 80. The sequence of motion of the arm and torso stretches the pectoral muscles 82 and rotates the shoulder 84

Returning to FIG. 6, and step 602, a number of example instructions are included depending on the sequence of motion. Where the sequence of motion is a foot during a walking/running/jogging step (see FIG. 7A, 72), the instructions direct a secondary user to frame the primary user's foot in the center of the viewfinder (in some embodiments, using a reticle), then follow the primary user as they take one or more steps with their foot.

In another example, where the target body part is a hand (see FIG. 7C), the UI instructs a single primary user to frame their relevant hand in the center of the viewfinder, and perform the requested sequence of motion (e.g., clenching and unclenching a fist, resting position to full extension of fingers, etc.). In a still further example, where the target body part is a breast (see FIG. 7A, 65), the instructions may include setting the camera on a table and performing aerobic activity in front of the camera (e.g., jumping jacks, walking/jogging/running toward the camera, etc.). In some embodiments, the instructions may include multiple videos. Differences between videos may include the variations in sequence of motion performed by the target body part and/or changes in position of the camera relative to the body part. Changes in position of the camera may be tracked by an internal inertial measurement unit (“IMU”) within the camera device (i.e., the IMU found on modern smartphone devices).

Techniques may be employed by the interface instructions to improve consistency. For example, auditory instructions about the current content of the viewfinder aids users who cannot see the viewfinder and do not have the aid of a secondary user (e.g., “take a small step to your left to center yourself in frame”). In another example, instructions direct the primary user to position themselves relative to a reference point that does not move despite repositioning of the camera (e.g., a mark on the ground, etc.). In some embodiments, the reference point may not be an intentionally chosen reference point. That is, the software onboard the camera may identify, via machine vision, a marking (e.g., such as a given point on a patterned floor, or a knot in a hardwood floor) and provide auditory instructions for the primary user to stand relative to that reference point without identifying the reference point to the user in the instructions.

In step 604, the processing server receives video data as collected from the mobile device. Once received, in step 606 and 608, the processing server performs machine vision operations on the acquired video data to determine size and curvature specifications for the user's applicable body part in different physical states (throughout the sequence of motion). In step 606, distances are mapped differently based on the manner in which the video data was gathered. In some embodiments, the video includes reference objects of known sizes. The reference object enables various other lengths to be derived.

In some embodiments, stereoscopic viewpoints can be used to identify distances/sizes. Numerous methods exist to obtain stereoscopic viewpoints. In some embodiments, some cameras include multiple lenses and naturally capture image data where derived depth is included in image meta data. In some single lens embodiments, where a secondary user operates the camera, the UI of the camera is instructed to shift the camera (as tracked by the IMU) prior to initiation of the sequence of movement. Frames captured during the initial shift of the camera enable the derivation of distances captured in later frames.

In some embodiments, a single user repositions the camera themselves and cannot guarantee consistent position of their body between the multiple stereoscopic viewpoints. In these embodiments, the camera may instead identify a reference points that is off the body of the primary user (e.g., static markings on the floor, heavy furniture, etc.). Reference points with a high certainty (determined via machine learning/machine vision) of static positioning are usable to determine a reference size in the video frames. Using the reference size, a remainder of distances and sizes included in the video data (including the target body part) may be derived.

In step 608, the processing server analyzes the video data for body part curvature and/or key points. The key points may exist both on and off the target body part (e.g., where a breast is the target body part, a key point may be on the user's breast and on the user's sternum). Key points that exist off the target body part are used as a control or reference point. The machine vision process seeks an expected curve or stress area associated with the body part and with the type of wearable selected. Key points and curves have corresponding data across a number of frames of the video data. In each frame, the key point has potentially shifted based on body part movement.

Once the curve or stress area is found, in step 610, points are plotted in 3-D space (either from video frames or static images) in a coordinate space. In some embodiments, the plotted points can be normalized for motion (e.g., correct for translational movement). Taken together, the collection of points model the transition the body part takes through the sequence of motion.

In step 610, the system identifies changes to each of the corresponding points across the video data. The data regarding the shift of a given key point illustrates where stress/force is being applied to the body, and how much force is present. Step 610 can be performed for any body part. However, as an example, a process for determining force/stress experienced by various portions of a breast during a sequence of motion is discussed below.

FIG. 8 is an illustration of a key point analysis on a female breast 65 during a sequence of motion. A number of “on-target body part” key points that can be tracked include a center breast point 86 (e.g., nipple), pectoral muscle points 88, outer key points 90, and inner key points 92. “Off-target body part” key points may include a sternum point 94 and/or an abdomen point 96.

Tracking the above points through the sequence of motion enables a determination of where stress is applied to the body and provides an approximation of the magnitude of that stress. For example, because force=(mass)(acceleration), force can be determined based on a derived value for breast mass and the acceleration of a given key point on the breast. Mass can be approximated using volume and density.

Volume can be calculated using derived dimensions (see FIG. 6, 606). Density can be approximated based on a breast stiffness criterion. Breast stiffness can be approximated based on the difference in movement of “on-target body part” key points (e.g., 86) and “off-target body part” key points (e.g., 94, 96). The difference in motion between the center of the breast and the sternum during aerobic activity can approximate the stiffness of the breast tissue. Using the breast stiffness and a statistical anatomical table, the system can derive an approximate breast density. From breast volume and breast density, the system can approximate breast mass.

The system calculates acceleration of various key points 86, 88, 90, 92 based on translational movement during the video data using the known length of the video data. Distance over time is velocity, and the derivative of velocity is acceleration. Thus, using the derived values for breast mass and acceleration at a given key point, the system computes the force experienced at that key point.

Calculating a nipple movement index (a combination of the displacement of the nipple and the acceleration of the nipple) based on the key points is a further statistic that may be used to evaluate a desirable support feature in a wearable.

Returning to FIG. 6, in step 612, the system incorporates secondary data sources. Examples of secondary data sources include the use of worn IMUs. For example, in some embodiments, the same mobile device used to capture the video data may subsequently be held by the user during a matching sequence of motion where acceleration is measured directly at the point the IMU is worn (e.g., on an armband, on an ankle band, held in the hand, etc.). The worn IMU data may support, supplement, or replace acceleration data otherwise derived. In some embodiments, the worn IMU is a device separate from the mobile device that captures the video data.

Other secondary sources of data include static image data (see FIGS. 4 and 5) and/or user preferences in the orthotics they wear. In step 614, the system identifies a stress and support profile to be included for the orthotic based on the force experienced by the body part relevant to the chosen orthotic, where that force is experienced, and the secondary data sources. A trained biometric model is applied to the collected and derived body part data to determine a style of wearable to generate that addresses that stress/support profile. In the example of FIG. 8, where the orthotic is a bra, the stress/support profile may call for varied bra strap configurations, bra strap thicknesses, padding thicknesses and positioning, and underwire configurations and thicknesses.

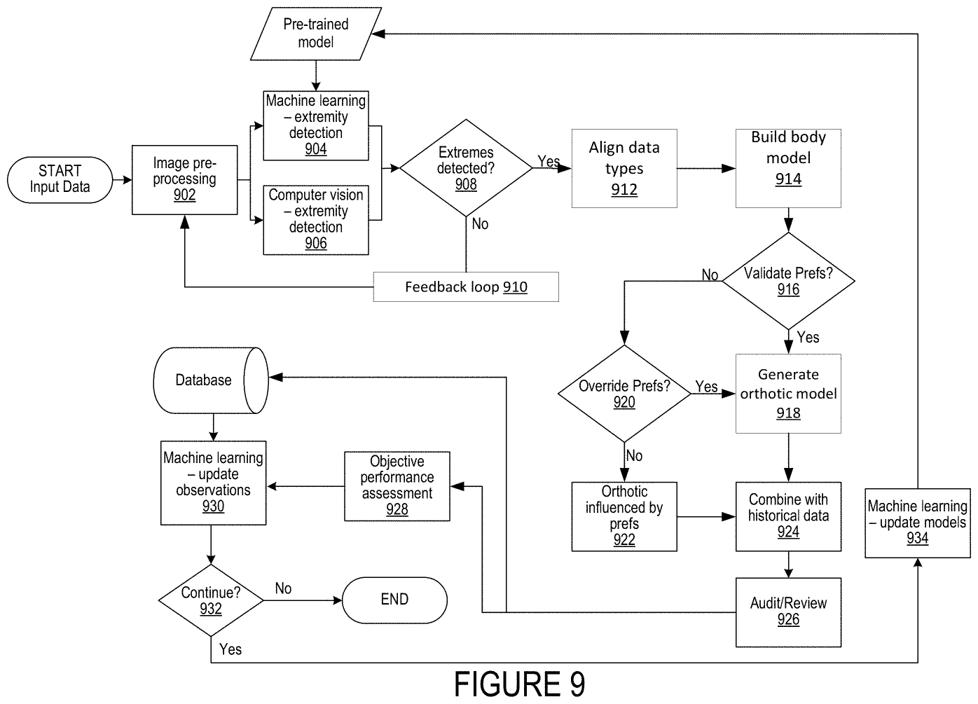

FIG. 9 is a flowchart illustrating wearable generation, including concurrent machine vision and machine learning processes on multiple data types. The steps of FIG. 9 are generally performed by the processing power available within the entire system. However, the processing power may be distributed across a number of devices and servers. For example, some steps (or modules) may be performed (or implemented) by a mobile device such as a smart phone while others are performed (or implemented) by a cloud server.

In FIG. 6, a video data source and secondary data sources are described. Each data type has advantages and disadvantages. Video data may not be as precise as static image data but does provide insight into physical transitions the body goes through while performing a sequence of movement. However, the disparate data sources cannot be inherently compared to one another.

In step 900, input body part image data is provided to the system. The input data may be provided in various ways (e.g., through direct upload from smartphone applications, web uploads, API uploads, partner application uploads, etc.). Initial input data describes a sequence of motion; examples may include uncategorized video frames, or a history of acceleration received by an IMU. The video frames/IMU data includes a known sequence of motion of a known target body part. “Uncategorized” refers to unknown physical conditions and weight loading states from frame to frame. Within a given sequence of motion, there are extremities (e.g., greatest weight loading/least weight loading, least to most extension, etc.). Identifying the frames where the body part reaches extremities enables the system to evaluate various sources of input with respect to one another. Data sources that are comparable construct better models than data that is evaluated in isolation. Static images (see FIGS. 4 and 5) include meta data that identifies the physical condition that the body part is under and often include static frames of the extremities. IMU data may be evaluated similarly as video data for extremities.

In step 902, the system prepares the sequence of motion data for categorization. In steps 904 and 906, the system detects the points during a sequence of motion where extremities are reached. This is performed both through computer vision and machine learning. For example, computer vision may analyze frames stepwise to identify where full extension of a body part is reached, whereas a trained machine learning model has a comparative background (the model training) for what a body part of the type being evaluated looks like when at a given extremity. Prior observations and models (e.g., a hidden Markov model) influence the machine learning operation.

In step 908, the system checks whether frames embodying the extremities are identified. Where system certainty is low, the method performs a feedback loop (810). In some embodiments, the user interface will additionally signal the user and the user may initiate the method again form the beginning. Where frames are identified as having extremities, the method proceeds to step 912.

In step 912, the system aligns disparate data sources to one another for comparison. Static images that are already labeled as extremity points are matched to those frames that are identified as extremities in steps 904 and 906. Static frames that include intermediate physical conditions (e.g., partial weight loading) are aligned with frames between the extremity frames.

In step 914, the system builds a model of the body part. In step 916, the system considers the user's preferences on worn orthotics. In some circumstances, the user's preferences are reflected in their body model. That is, their preferences are consistent with what is recommended based on their anatomy. Where the preferences are consistent, in step 918, a 3-D model of the orthotic is generated according to model recommendation and the rest of the orthotic printing process continues separately.

Where the user's preferences are not validated by the body model, in step 920, the system determines whether to override the user's preferences. Overriding a user's preferences is based on a degree of deviation implementing the user's preferences in the orthotic would cause to an orthotic built based purely on a recommendation using the body model of step 914. Where the degree of deviation is below a threshold, the method proceeds to step 922, and generates an orthotic model that is influenced by the user's preferences.

Where the degree of deviation is above a threshold, the user may be queried regarding their preferences. Where the user is insistent on their preferences, the method similarly proceeds to step 922. Where the threshold is exceeded, and the user does not insist upon implementation of their preference, the method proceeds to step 918, and generates an orthotic according to model recommendation. The rest of the orthotic printing process continues separately.

In step 924, the system adds the transmitted images to the total observations. In step 926, the system enables users, or administrators to do an audit review.

After steps 924-926, the data is added to a database. The process of FIG. 9 continues with an assessment and learning phase. In step 928, the system reviews and performs a performance assessment of the process. In step 930, the machine learning engine of the system updates the observations from the database and the performance assessment. If the process continues, in step 934, the machine leaning models are updated. The updated machine learning models are recycled into use into step 904 for subsequent users (e.g., through application updates or API updates).

Although the invention is described herein with reference to the preferred embodiment, one skilled in the art will readily appreciate that other applications may be substituted for those set forth herein without departing from the spirit and scope of the present invention. Accordingly, the invention should only be limited by the claims included below.

1. A method comprising:
receiving, through a mobile device, image data of a body part, wherein the body part is under a plurality of physical conditions;
generating digital models of the body part under the plurality physical conditions based on the image data;
generating a 3D model of a wearable object corresponding to the body part, wherein the 3D model is based on at least one of the digital models; and
sending, for delivery to a manufacturing apparatus, print instructions including information associated with generating a physical wearable object based on the 3D model of the wearable object.

2. The method of claim 1, wherein the image data includes a plurality of video frames of the body part and the plurality of physical conditions includes execution of a sequence of motion.

3. The method of claim 2, wherein the body part is a foot, and the sequence of motion is execution of a walking or running step, the method further comprising:
identifying a style of foot arch alteration throughout the course of the execution of the step by using a plurality of tracked key points across the plurality of video frames of the body part.

4. The method of claim 2, wherein the body part is a breast, and the sequence of motion is a shift of the breast during another body movement, the method further comprising:
mapping a 3D breast shift using a plurality of tracked key points across the plurality of video frames of the body part.

5. The method of claim 2, wherein the body part is a breast, and the sequence of motion is a shift of the breast during another body movement, the method further comprising:
calculating a breast stiffness based on a tracked on-breast key point and a tracked off-breast key point.

6. The method of claim 2, wherein the body part is a breast, and the sequence of motion is a shift of the breast during another body movement, the method further comprising:
calculating a nipple movement index including a nipple displacement and a nipple acceleration, the nipple movement index based on a tracked on-breast key point and a tracked off-breast key point.

7. The method of claim 5, further comprising:
calculating a breast mass based on the breast stiffness and a breast size, the breast size calculated using either: a) a reference object in the plurality of frames from a video of the body part or b) stereoscopic images from the plurality of frames.

8. The method of claim 7, further comprising:
calculating a magnitude of breast inertia based on a plurality of tracked key points across the plurality of video frames of the body part and the breast mass.

9. The method of claim 2, further comprising:
displaying, on the mobile device, instructions that describe the sequence of motion of the body part.

10. The method of claim 2, wherein the body part is any of:
a single joint;
a foot;
a head and neck;
a hand; or
a breast.

11. The method of claim 1, wherein the plurality of physical conditions include any of:
supporting different weight loads;
flexed and unflexed;
clenched and unclenched;
at different states of gravity; or
at different temporal periods.

12. The method of claim 1, further comprising:
receiving user input regarding wearable object preference, wherein said generating the 3D model of a wearable object corresponding to the body part is further based on the user input.

13. The method of claim 1, wherein the image data of the body part includes both 2D image data and 3D image data.

14. The method of claim 1, further comprising:
receiving motion data from a sensor worn on a body during a sequence of motion of the body part, wherein said generating the 3D model of a wearable object corresponding to the body part is further based on the motion data.

15. The method of claim 13, wherein the sensor is an accelerometer of the mobile device, and the motion data comprises a magnitude of force generated.

16. A method comprising:
receiving, via a mobile device, body part data include two or more of:
video data of a sequence of motion of a body part;
2D static image data of the body part under a plurality of physical conditions;
3D static image data of the body part under the plurality of physical conditions;
user preferences regarding a wearable item; or
inertial measurement unit data associated with the sequence of motion of the body part;
generating a digital model of the body part based on the body part data;
generating a 3D model of a wearable object corresponding to the body part, wherein the 3D model is based on the digital model; and
sending, for delivery to a manufacturing apparatus, print instructions including information associated with generating a physical wearable object based on the 3D model of the wearable object.

17. The method of claim 16, wherein the body part is a breast and the method further comprising:
calculating a breast stiffness based on a tracked on-breast key point and a tracked off-breast key point identified from the video data of a sequence of motion of a body part.
calculating a breast size based on inclusion of a reference object or stereoscopic image frames from any of: the video data of the sequence of motion of the body part, the 2D static image data, or the 3D static image data;
calculating a breast mass based on the breast stiffness and the breast size; and
calculating a magnitude of breast inertia based on the breast mass and the inertial measurement unit data.

18. The method of claim 16, wherein the body part is a foot, the sequence of movement is an execution of a step, and the method further comprising:
identifying a style of foot arch alteration throughout the execution of the step using a plurality of tracked key points across a plurality of frames of the video data.

19. The method of claim 16, wherein the body part is a knee, and the plurality of physical conditions include supporting full body weight, and supporting no body weight, the method further comprising:
identifying a shift in key points between the plurality of physical conditions.

20. A system comprising:
a camera of a mobile device, configured to capture image data of a body part under a plurality of physical conditions;
an inertial measurement unit of the mobile device, configured to acquire motion data during a sequence of motion by the body part; and
a processor configured to generate a digital model of the body part based on the image data and the motion data, and further configured to generate a 3D model of a wearable object corresponding to the body part based on the digital model.

21. The system of claim 20, wherein the plurality of physical conditions include any of:
supporting different weight loads;
flexed and unflexed;
clenched and unclenched;
at different states of gravity; or
at different temporal periods.

22. The system of claim 20, further comprising:
a display configured to display mobile device positioning information associated with collection of the image data and motion data.

23. The system of claim 20, further comprising:
a network transceiver configured to transmit print instructions for delivery to a manufacturing apparatus, the print instructions including information associated with generating a physical wearable object based on the 3D model of the wearable object.

24. A system comprising:
a camera of a mobile device, configured to capture image data of a body part under a plurality of physical conditions; and
a processor configured to process motion data during a sequence of motion by the body part and generate a digital model of the body part based on the image data and the motion data, and further configured to generate a 3D model of a wearable object corresponding to the body part based on the digital model.

25. The system of claim 24, wherein the plurality of physical conditions include any of:
supporting different weight loads;
flexed and unflexed;
clenched and unclenched;
at different states of gravity; or
at different temporal periods.

26. The system of claim 24, further comprising:
a display configured to display mobile device positioning information associated with collection of the image data and motion data.

27. The system of claim 24, further comprising:
a network transceiver configured to transmit print instructions for delivery to a manufacturing apparatus, the print instructions including information associated with generating a physical wearable object based on the 3D model of the wearable object.
